(19) United States
(12) Patent Application Publication
(10) Pub. No.: US 2010/0006103 A1
(43) Pub. Date: Jan. 14, 2010

(54) ENDOTRACHEAL-OXIMETER DEVICE, SYSTEM AND METHOD OF USING SAME

(76) Inventors: William J. McGinnis, Cincinnati, OH (US); Scott A. METRICK, Glenview, IL (US)

Correspondence Address: LADAS & PARRY LLP, 224 SOUTH MICHIGAN AVENUE, SUITE 1600, CHICAGO, IL, 60604, US

(21) Appl. No.: 12/170,535

(22) Filed: Jul. 10, 2008

(51) Int. Cl. A61M 16/04

(52) U.S. Cl. 128/207.15

(57) ABSTRACT

An endotracheal-oximeter device is presented which includes a flexible conduit having a pair of light emitters, a detector, a control circuit, a display unit and a power source attached to the conduit. The flexible conduit has an airway passing between an upper and lower end. The detector is mounted directly onto the conduit so that the detector is already optically aligned to receive the first and second EMF emissions from the pair of light emitters so as to produce a detector signal in response to the received first and second EMF emissions. The endotracheal-oximeter system includes an endotracheal tube and a monitoring station. The endotracheal tube of the system is composed of the flexible conduit having the pair of light emitters, the detector, the control circuit, the power source and a transmitter. The monitoring station of the system includes an antenna, a receiver circuit, and the display unit. The method of using includes the steps of activating, aligning, enabling, forcing, getting, inspecting, obtaining, securing and sliding.

### FIELD OF THE INVENTION

The present invention relates to surgical devices, medical treatment and diagnosis of patients. More particularly to an endotracheal-oximeter device configured to have a prealigned oximeter mounted on the endotracheal tube for providing estimates of oxygen levels in patients during intubation.

### BACKGROUND OF THE INVENTION

Emergency medical treatment occasionally requires endotracheal intubation to provide an unobstructed air passageway to the patient's lungs to assure adequate aeration. Unfortunately during emergency medical situations, correct positioning of the endotracheal tube may be overlooked. As a result of failed intubation, death, brain injury, airway trauma, tracheal or esophageal perforation, pneumothorax and aspiration have been known to occur.

It is also standard practice to insert an endotracheal tube through the patient's mouth and into the trachea to connect the patient to a ventilator to assist breathing while conducting surgery under general anesthesia. Even if an endotracheal tube is initially positioned correctly, it is still prone to movement and misalignment within the patient's trachea. This poses a continual potential problem because misaligned endotracheal tubes can contribute to death, brain injury, airway trauma, tracheal or esophageal perforation, pneumothorax and aspiration have been known to occur.

Oximetry provides a convenient non-invasive way of estimating oxygen levels in patients. Oximetry is based on exploiting the different light absorptive physical properties of oxygenated and non-oxygenated hemoglobins. It is believed that oxygenated hemoglobin absorbs red light more strongly than infrared radiation. Conversely, it is believed that non-oxygenated heomoglobin absorbs infrared radiation more strongly than red light. This differential absorptive behavior is the basis of oximetry for providing a convenient non-invasive measurement of oxygen levels in arterial blood and surrounding tissue.

An endotrachael tube coupled to a built in oximeter would prove to be useful in providing a convenient means for estimating oxygen in the patient which could provide a secondary indication that the endotrachael tube is positioned correctly within the patient's trachea.

While presently known endotracheal tubes fulfill many of their respective objectives and requirements, no endotracheal tubes or devices describe a flexible conduit, first and second EMF emitters, a detector mounted directly onto the conduit so that the detector is optically aligned to receive the first and second EMF emissions, a control circuit, a display unit and a power source. This combination of interconnected elements would specifically match the user's particular individual needs of making it possible to provide a means for treating patient with an endotracheal tube and diagnosing oxygen levels of the patient using a conveniently aligned oximeter mounted onto the endotracheal tube.

Therefore, a need exists for a new and improved endotracheal-oximeter device, system and method for use in providing estimates of oxygen levels during intubation. In this respect, the endotracheal-oximeter device according to the present invention substantially departs from the conventional concepts and designs of the prior art, and in doing so provides an apparatus primarily developed for the purpose of providing a convenient means for obtaining estimates of oxygen levels during intubation.

### SUMMARY OF THE INVENTION

The present device, system and method of using, according to the principles of the present invention, overcomes a number of the shortcomings of the prior art by providing a novel endotracheal-oximeter device, system and method for use in providing estimates of oxygen levels during intubation. The endotracheal-oximeter device includes a flexible conduit having a pair of EMF emitters, a detector, a control circuit, a display unit and a power source attached to the conduit. The flexible conduit has an airway passing between an upper and lower end. The detector is mounted directly onto the conduit so that the detector is already optically aligned to receive the first and second EMF emissions from the pair of EMF emitters so as to produce a detector signal in response to the received first and second EMF emissions. The endotracheal-oximeter system includes an endotracheal tube and a monitoring station. The endotracheal tube of the system is composed of the flexible conduit having the pair of light emitters, the detector, the control circuit, the power source and a transmitter. The monitoring station of the system includes an antenna, a receiver circuit, and the display unit. The method of using includes the steps of activating, aligning, enabling, forcing, getting, inspecting, obtaining, securing and sliding.

In view of the foregoing disadvantages inherent in the known type endotracheal tube devices now present in the prior art, the present invention provides an improved endotracheal-oximeter device, which will be described subsequently in great detail, is to provide a new and improved endotracheal-oximeter device which is not anticipated, rendered obvious, suggested, or even implied by the prior art, either alone or in any combination thereof.

To attain this, the present device embodiment of the invention essentially comprises the interconnected components of a flexible conduit, a first electromagnetic force (EMF) emitter, a second EMF, a detector, a control circuit, a display unit, and a power source. The flexible conduit has an upper end, a lower end and an airway that passes between the upper and lower ends. The first EMF emitter is attached to the conduit in which the first EMF emitter is configured to emit a first EMF emission. The second EMF emitter is attached to the conduit in which the second EMF emitter is configured to emit a second EMF emission. The detector is mounted directly onto the conduit so that the detector is optically aligned to receive the first and second EMF emissions. The detector is configured to produce a detector signal in response to the received first and second EMF emissions. The control circuit is attached to the conduit in which the control circuit is electrically coupled to the first EMF emitter, to the second EMF emitter and to the detector. The display unit is attached to the conduit and is electrically coupled to the control circuit. The display unit is configured to provide an estimate of oxygen saturation in tissue in response to the detector signal. The power source is attached to the conduit in which the power source is electrically coupled to the control circuit.

To attain this, the present system embodiment of the invention essentially comprises an endotracheal tube and a monitor station. The endotracheal tube comprises a flexible conduit, a first EMF emitter, a second EMF, a detector, a control circuit, a power source, and a transmitter. The flexible conduit has an upper end, a lower end, and an airway that passes between the upper end and the lower end. The first EMF emitter is attached to the conduit in which the first EMF emitter is configured to emit a first EMF emission. The second EMF emitter is attached to the conduit in which the second EMF emitter is configured to emit a second EMF emission. The detector is mounted directly onto the conduit so that the detector is optically aligned (either directly or indirectly) to receive the first and second EMF emissions. The detector is configured to produce a detector signal in response to the received first and second EMF emissions. The control circuit is attached to the conduit in which the control circuit is electrically coupled to the first EMF emitter, to the second EMF emitter, and to the detector. The power source is attached to the conduit in which the power source is electrically coupled to the control circuit. The transmitter is attached to the conduit and is electrically coupled to the control circuit. The transmitter is configured to transmit a broadcast signal proportionate to the detector signal from the control circuit.

To attain this, the present method embodiment of the invention essentially comprises steps of activating, aligning, enabling, forcing, getting, inspecting, obtaining, securing and sliding.

There has thus been outlined, rather broadly, the more important features of the invention in order that the detailed description thereof that follows may be better understood, and in order that the present contribution of the art may be better appreciated.

Numerous aspects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved endotracheal-oximeter device that has many of the advantages of the prior endotracheal tube devices and minimizes a number of their disadvantages.

It is another aspect of the present invention to provide a new and improved endotracheal-oximeter device that may be easily and efficiently manufactured and marketed.

An even further aspect of the present invention is to provide a new and improved endotracheal-oximeter device that has a low cost of manufacture with regard to both materials and labor, and which accordingly is then susceptible of low prices of sale to the consuming public, thereby making the endotracheal-oximeter device economically available to the buying public.

Still another aspect of the present invention is to provide a endotracheal-oximeter device that provides some of the advantages over basic endotracheal tube devices, while simultaneously overcoming some of the disadvantages normally associated therewith.

Even still another aspect of the present invention is to provide a endotracheal-oximeter device having the interconnected components such that the attached detector is mounted directly onto the conduit so that the detector is already optically aligned to receive the first and second EMF emissions from the pair of EMF emitters so as to produce a detector signal in response to the received first and second EMF emissions.

Yet still another aspect of the present invention is to provide a system composed of endotracheal-oximeter tube which is in radio communications with a monitor station.

Lastly, it is an aspect of the present invention to provide a new and improved method of using comprising the steps of activating, aligning, enabling, forcing, getting, inspecting, obtaining, securing and sliding.

There has thus been outlined, rather broadly, the more important features of the invention in order that the detailed description thereof that follows may be better understood, and in order that the present contribution of the art may be better appreciated.

Numerous other features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other aspects of the invention, along with the various features of novelty that characterize the invention, are pointed out with particularity in the claims annexed to and forming a part of this disclosure. For a better understanding of the invention, its operating advantages and the specific aspects attained by its uses, reference should be had to the accompanying drawings and description matter in which there are illustrated preferred embodiments of the invention.

### BRIEF DESCRIPTION OF THE DRAWINGS

The invention will be better understood and aspects other than those set forth above will become apparent when consideration is given to the following detailed description thereof. Such description makes reference to the annexed drawings wherein:

FIG. 1A is a side view of an embodiment of the endotracheal-oximeter device constructed in accordance with the principles of the present invention;

FIG. 1B a side view of another embodiment of the endotrachael-oximeter device of the present invention;

FIG. 2A is a partial cross sectional view of an another embodiment of the endotracheal-oximeter device of the present invention shown functioning in the reflectance oximeter operational measurement mode;

FIG. 2B is a partial cross sectional view of another embodiment of the endotracheal-oximeter device of the present invention shown functioning in the transmittance oximeter operational measurement mode;

FIG. 3 is a perspective side view of an another embodiment of the endotrachael-oximeter device of the present invention shown placed within a trachea;

FIG. 4 is a partial cut away side view of an embodiment of the endotracheal-oximeter system of the present invention placed in a trachae;

FIG. 5 is a partial cross-sectional view of the conduit of another embodiment of the endotracheal-oximeter system of the present invention;

FIG. 6 is a side view of an embodiment of the endotracheal-oximeter system of the present invention; and

FIG. 7 is a perspective view of a monitoring station of an embodiment of the endotracheal-oximeter system of the present invention.

The same reference numerals refer to the same parts throughout the various figures.

### DETAILED DESCRIPTION OF THE PREFERRED EMBODIMENT

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

Accordingly, the detailed discussion herein of one or more embodiments is not intended, nor is to be construed, to limit the metes and bounds of the patent protection afforded the present invention, in which the scope of patent protection is intended to be defined by the claims and their equivalents thereof. Therefore, embodiments not specifically addressed herein, such as adaptations, variations, modifications, and equivalent arrangements, should be and are considered to be implicitly disclosed by the illustrative embodiments and claims described herein and therefore fall within the scope of the present invention.

Further, it should be understood that, although steps of various the claimed method may be shown and described as being in a sequence or temporal order, the steps of any such method are not limited to being carried out in any particular sequence or order, absent an indication otherwise. That is, the claimed method steps are to be considered to be capable of being carried out in any sequential combination or permutation order while still falling within the scope of the present invention.

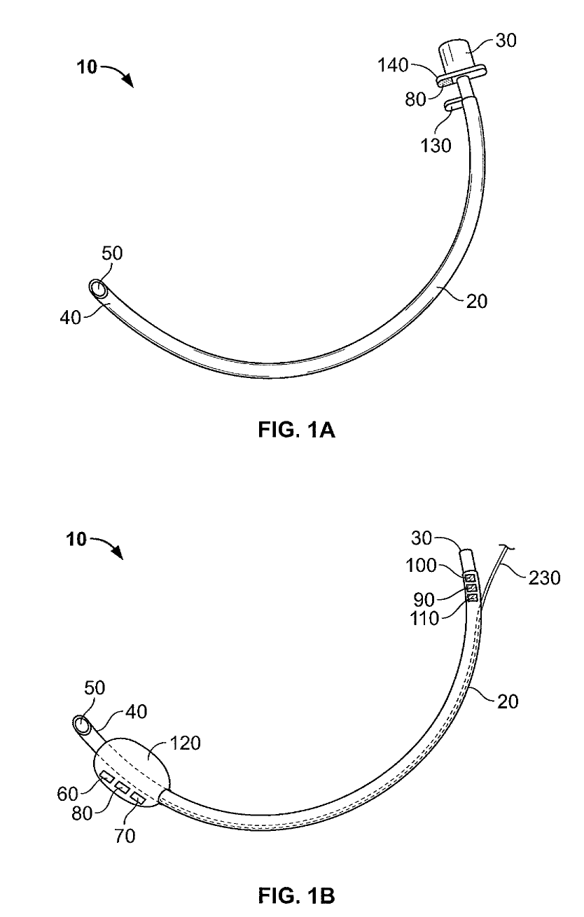

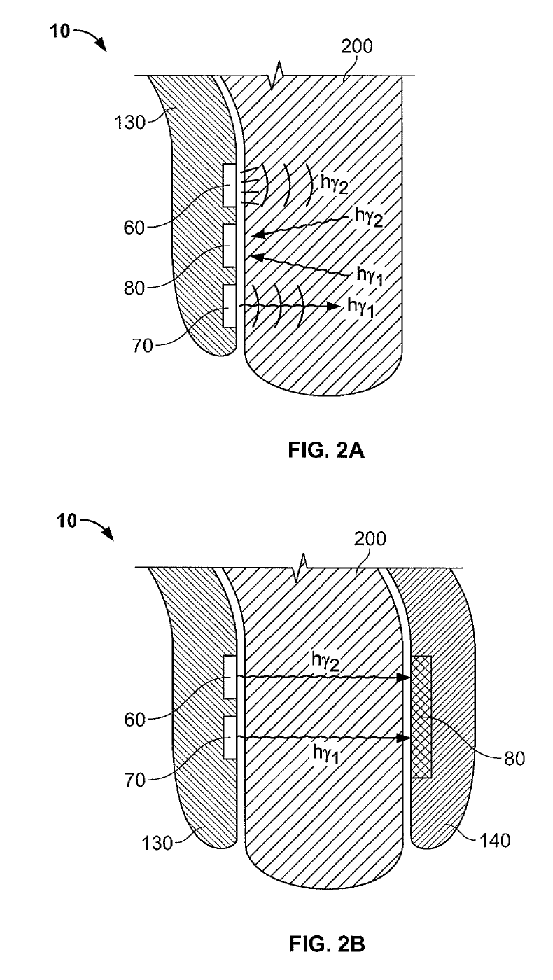

Referring now to the drawings, and in particular FIGS. 1 to 7 thereof, one preferred embodiment of the present invention is shown and generally designated by the reference numeral 10. One preferred embodiment of An endotracheal-oximeter device 10 may include a flexible conduit 20, a first electromagnetic force (EMF) emitter, a second EMF emitter 70, a detector 80, a control circuit 90, a display unit 100, and a power source 110. The flexible conduit 20 has an upper end 30, a lower end 40 and an airway 50 that passes between the upper and lower end 40s. The first EMF emitter 60 is attached to the conduit 20 in which the first EMF emitter 60 is configured to emit a first EMF emission. The second EMF emitter 70 is attached to the conduit 20 in which the second EMF emitter 70 is configured to emit a second EMF emission. The detector 80 is mounted directly onto the conduit 20 so that the detector 80 is optically aligned to receive the first and second EMF emissions. The detector 80 is configured to produce a detector signal in response to the received first and second EMF emissions. The control circuit 90 is attached to the conduit 20 in which the control circuit 90 is electrically coupled to the first EMF emitter 60, to the second EMF emitter 70 and to the detector 80. The display unit 100 is attached to the conduit 20 and is electrically coupled to the control circuit 90. The display unit 100 is configured to provide an estimate of oxygen saturation in tissue 200 in response to the detector signal. The power source 110 is attached to the conduit 20 in which the power source 110 is electrically coupled to the control circuit 90.

The conduit 20 may have any known geometry so long as the lower end 40 of the conduit 20 can be slid down into the trachae 210 of a patient 220. One variation of the conduit 20 is that the conduit 20 has an inflatable cuff 120. The inflatable cuff 120 can be inflated by an inflating tube 230.

The first and second EMF emitters (60 and 70) and the detector 80 can be located anywhere along the device 10 as long as the detector 80 is optically aligned to receive the first and second EMF emissions from their respective first and second EMF emitters (60 and 70). The optical alignment can be direct or indirect. Direct optical alignment shall be understood to mean that the first and second EMF emitters (60 and 70) are each lined up with the detector 80 so that a substantial portion of their respective emissions can be shone directly onto the detector 80. Indirect optical alignment shall be understood to mean that the first and second EMF emitters (60 and 70) are not physically inline with the detector 80. Accordingly in indirect optical alignment no substantial portion of the respective emissions of the first and second EMF emitters (60 and 70) is expected to directly reach the detector 80. Rather in indirect optical alignment only those respective emissions of the first and second EMF emitters (60 and 70) that are somehow back-reflected or backscattered are expected to reach the detector 80 in substantial measurable quantities.

One preferred placement of the first and second EMF emitters (60 and 70) and the detector 80 is that they are attached to the inflatable cuff 120 of the conduit 20. This preferred placement is intended to allow the first and second EMF emitters (60 and 70) and the detector 80 to function in a reflectance oximeter operational mode.

Another preferred placement of the first and second EMF emitters (60 and 70) and the detector 80 is on a ridge 130 of the conduit 20. That is, the first EMF emitter 60, the second EMF emitter 70, and the detector 80 are all attached to the ridge 130 of the conduit 20. This preferred placement is also intended to allow the first and second EMF emitters (60 and 70) and the detector 80 to function in a reflectance oximeter operational mode.

Yet another preferred placement of the first and second EMF emitters (60 and 70) and the detector 80 is on a ridge 130 and an opposing protrusion 140 of the conduit 20. That is, the first and second EMF emitters (60 and 70) may be attached to the ridge 130 and the detector 80 may be attached to the opposing protrusion 140. Alternately, the first and second EMF emitters (60 and 70) may be attached to the opposing protrusion 140 and the detector 80 may be attached to the ridge 130. These preferred placements are intended to allow the first and second EMF emitters (60 and 70) and the opposing detector 80 to function in a transmittance oximeter operational mode. It is understood that the attending health care worker or physician could be able to place a portion of tissue 200, such as a patient 220's lip, between the ridge 130 and protrusion 140 so that the oxygen saturation levels of the patient 220 can be subsequently estimated from the patient 220.

Still yet another preferred placement of the first and second EMF emitters (60 and 70) and the detector 80 is on either a ridge 130 or an opposing protrusion 140 of the conduit 20. That is, the first and second EMF emitters (60 and 70) and the detector 80 may be attached to the ridge 130 of the conduit 20. Alternately, the first and second EMF emitters (60 and 70) and the detector 80 may be attached to the opposing protrusion 140 of the conduit 20. These preferred placements are intended to allow the first and second EMF emitters (60 and 70) and the detector 80 to function in a reflectance oximeter operational mode. It is understood that the attending health care worker or physician could be able to restrain a portion of tissue 200, such as a patient 220's lip, between the ridge 130 and protrusion 140 so that the oxygen saturation levels of the patient 220 can be subsequently estimated from the patient 220.

The particular placement of where the control circuit 90 and the power source 110 may be located almost anywhere on the conduit 20. One preferred embodiment is that the control circuit 90 and the power source 110 are sequestered within the wall of the conduit 20. Another preferred embodiment is that the control circuit 90 and the power source 110 are positioned near the upper end 30 of the conduit 20 for easy access.

The first and second EMF emitters (60 and 70) may be any type of known type of EMF emitters such as being light emitting diodes (LEDs) or lasers. Accordingly, the first and second EMF emissions may either emit broadband EMF emissions or monochromatic EMF emissions. One preferred embodiment of the first EMF emitter 60 is that it is configured to emit the first EMF emission having a red frequency emission between about 625 to about 740 nanometers. A more preferred embodiment is that the first EMF emitter 60 is configured to emit the first EMF emission that has a mean red emission frequency at about 660 nanometers. One preferred embodiment of the second EMF emitter 70 is that it is configured to emit the second EMF emission at an infrared frequency between about 800 to about 1100 nanometers. A more preferred embodiment of the second EMF emitter 70 is configured to emit the second EMF emission having a mean infrared emission frequency at about 940 nanometers.

The detector 80 may be any known type of detector 80 such as those selected from the group consisting of a photodetector, a photodiode, a pin diode, a phototransistor, a charge-coupled device 10 (CCD) array, and a photomultiplier tube. Further to reduce extraneous unwanted light, the detector 80 may also be equipped with a spectral bandpass filter for selectively enhancing the signal to noise sensitivity of the resultant incoming signal. The detector 80 may be mounted almost anywhere along the device 10 as long as it is optically coupled (directly or indirectly) to the first and second EMF emitters (60 and 70). One preferred embodiment is that the detector 80 is mounted onto the conduit 20 so that detector 80 is configured to directly detect the first and second EMF emissions transmitted through tissue 200 in accordance to a transmittance oximetry measurement scheme. Another preferred embodiment is that the detector 80 is mounted onto the conduit 20 so that detector 80 is configured to detect the first and second EMF emissions scattered by tissue 200 in accordance to a reflectance oximetry measurement scheme.

The display unit 100 may be any type of display unit 100 as long as it is configured to provide an indication of an estimate of oxygen saturation in blood. Accordingly, some preferred embodiments of the display unit 100 can be selected from the group consisting of a light emitting diode display, an incandescent light display, a computer terminal display, and an audible alarm display. One variation of the preferred embodiment of the audible display unit 100 is that it is configured to produce an audible alarm when the estimate of oxygen saturation in blood is below a minimum threshold value.

The power source 110 attached to the conduit 20 may be any commercially available power source 110 such as those selected from the group consisting of a battery power source 110, a high capacity capacitor power source 110, and an electrical wire configured to be connected to a wall socket.

One preferred embodiment of the endotracheal-oximeter system 150 comprises: an endotracheal tube 240 and a monitor station 170. The endotracheal tube 240 comprises a flexible conduit 20, a first EMF emitter 60, a second EMF, a detector 80, a control circuit 90, a power source 1101 and a transmitter 160. The flexible conduit 20 has an upper end 30, a lower end 40, and an airway 50 that passes between the upper end 30 and the lower end 40. The first EMF emitter 60 is attached to the conduit 20 in which the first EMF emitter 60 is configured to emit a first EMF emission. The second EMF emitter 70 is attached to the conduit 20 in which the second EMF emitter 70 is configured to emit a second EMF emission. The detector 80 is mounted directly onto the conduit 20 so that the detector 80 is optically aligned (either directly or indirectly) to receive the first and second EMF emissions. The detector 80 is configured to produce a detector signal in response to the received first and second EMF emissions. The control circuit 90 is attached to the conduit 20 in which the control circuit 90 is electrically coupled to the first EMF emitter 60, to the second EMF emitter 70, and to the detector 80. The power source 110 is attached to the conduit 20 in which the power source 110 is electrically coupled to the control circuit 90. The transmitter 160 is attached to the conduit 20 and is electrically coupled to the control circuit 90. The transmitter 160 is configured to transmit a broadcast signal proportionate to the detector signal from the control circuit 90. The monitor station 170 comprises an antenna 180, a receiver circuit 190 and a display unit 100. The antenna 180 is configured to receive the broadcast signal transmitted from the transmitter 160. The receiver circuit 190 is electrically coupled to the antenna 180 in which the receiver circuit 190 is configured to process the received broadcast signal into a processed signal. The display unit 100 is operatively coupled to the receiver circuit 190 in which the display unit 100 is configured to display the processed signal which can be used by an attending health care person to use as an estimate of oxygen saturation in tissue 200.

One preferred embodiment of a method of using an endotracheal-oximeter system 150 comprises the steps of activating, aligning, enabling, forcing, getting, inspecting, obtaining, securing, and sliding. The obtaining step comprises obtaining an endotracheal tube 240 comprising: a flexible conduit 20 comprising an upper end 30, a lower end 40, and an airway 50 between the upper end 30 and the lower end 40; a first electromagnetic force (EMF) emitter attached to the conduit 20 wherein the first EMF emitter 60 is configured to emit a first EMF emission; a second EMF emitter 70 attached to the conduit 20 wherein the second EMF emitter 70 is configured to emit a second EMF emission; a detector 80 mounted directly onto the conduit 20 so that the detector 80 is optically aligned to receive the first and second EMF emissions, wherein the detector 80 is configured to produce a detector signal in response to the received first and second EMF emissions; a control circuit 90 attached to the conduit 20, wherein the control circuit 90 is electrically coupled to the first EMF emitter 60, to the second EMF emitter 70, and to the detector 80; and a power source 110 attached to the conduit 20, wherein the power source 110 is electrically coupled to the control circuit 90; a transmitter 160 attached to the conduit 20 and electrically coupled to the control circuit 90, wherein the transmitter 160 is configured to transmit a broadcast signal proportionate to the detector signal from the control circuit 90. The getting step comprises getting a monitor station 170 comprising: an antenna 180 configured to be receive the broadcast signal transmitted from the transmitter 160; a receiver circuit 190 electrically coupled to the antenna 180, the receiver circuit 190 is configured to process the received broadcast signal into a processed signal; and a display unit 100 operatively coupled to the receiver circuit 190, the display unit 100 is configured to display the processed signal which can be used as an estimate of oxygen saturation in tissue 200. The activating step comprises activating the control circuit 90 with the power source 110. The activating step results in the first and second EMF emitters (60 and 70) emitting their respective first and second EMF emissions so that the detector 80 senses the emitted first and second EMF emissions and results in the transmitter 160 transmitting the broadcast signal proportionate to the detector signal from the control circuit 90. The aligning step comprises aligning a patient 220 to receive the endotracheal tube 240. The sliding step comprises sliding the lower end 40 of the conduit 20 of the endotracheal tube 240 down into the trachae 210 of the patient 220. The securing step comprises securing the endotrachael tube 240 in the trachae 210 of the patient 220. The forcing step comprises forcing air through the airway 50 of the conduit 20 of the secured endotracheal tube 240 to aerate the patient 220. The enabling step comprises enabling the monitor station 170. The enabling step enables the antenna 180 to receive the broadcast signal transmitted from the transmitter 160 so that the receiver circuit 190 subsequently processes the received broadcast signal into a processed signal and display unit 100 displays the processed signal. The inspecting step comprises inspecting the displayed processed signal by using the displayed processed signal as an estimate of oxygen saturation in tissue 200 of the patient 220.

FIG. 1A depicts a side view of an embodiment of the endotracheal-oximeter device 10 having a flexible conduit 20, a ridge 130 and an opposing protrusion 140. The flexible conduit 20 is shown having an upper end 30, a lower end 40, and an airway 50 that passes between the upper end 30 and the lower end 40. The protrusion 140 is shown having a detector 80. The first and second EMF emitters (60 and 70) (not shown) are mounted onto the ridge 130 so that their respective first and second EMF emissions are directly aligned with the detector 80. In this configuration the endotracheal-oximeter is intended to function in the transmittance oximeter operational mode. Accordingly an attending health care worker could conveniently position a portion of tissue 200 (not shown), such as a tongue or a lip, across the ridge 130 and the opposing protrusion 140 to obtain an estimate of the oxygen saturation levels of the patient 220 (not shown).

FIG. 1B depicts a side view of another embodiment of the endotrachael-oximeter having a flexible conduit 20, the first and second EMF emitters (60 and 70), the detector 80, the control circuit 90, the display unit 100 and the power source 110. The flexible conduit 20 is shown having an upper end 30, a lower end 40, and an airway 50 that passes between the upper end 30 and the lower end 40. The conduit 20 is shown having an inflatable cuff 120 and an inflating tube 230. The first and second EMF emitters (60 and 70) and detector 80 are shown mounted onto the cuff 120 so that when the cuff 120 is inflated to secure the conduit 20 in the trachae 210 (not shown) of then the first and second EMF emitters (60 and 70) and detector 80 are in intimate contact with adjacent tissue 200. In this configuration the endotracheal-oximeter device 10 is intended to function in the reflectance oximeter operational mode. Accordingly an attending health care worker could conveniently obtain an estimate of the oxygen saturation levels of the patient 220 (not shown) by inspecting the display unit 100 at the upper end 30 of the conduit 20.

FIG. 2A depicts a partial cross-sectional view of another embodiment of the endotracheal-oximeter device 10 that is intended to function in a reflectance oximeter operational mode. In particular FIG. 2A shows that the first and second EMF emitters (60 and 70) and the detector 80 are mounted onto a common surface of a ridge 130 and pointing away from each other. That is the first and second EMF emitters (60 and 70) are not linearity aligned with the detector 80 and thus this geometric placement configuration is intended to function in the reflectance oximeter operational mode. Radiant energy (hv₁ and hv₂) is shown emitted from the first and second EMF emitters (60 and 70) into an adjacently placed tissue 200, e.g., a lip of a patient 220. This emitted energy is scattered throughout the tissue 200 of the patient 220 in which some of this scattered emitted energy is back scattered towards the detector 80. The detector 80 subsequently detects a portion of this back scattered emitted radiant energy and consequently produces a detector signal which can subsequently be used to estimate the blood oxygen level in the tissue 200.

FIG. 2B depicts a partial cross-sectional views of another embodiment of the endotracheal-oximeter device 10 shown functioning in the transmittance oximeter operational measurement mode. The first and second EMF emitters (60 and 70) are shown mounted onto the ridge 130 and the detector 80 is shown mounted onto the protrusion 140. Accordingly, the first and second EMF emitters (60 and 70) and the detector 80 oppose each other and thus this geometric placement configuration is intended to function in the transmittance operational mode. Radiant energy (hv₁ and hv₂) is shown emitted from the first and second EMF emitters (60 and 70) and into an adjacently placed tissue 200, e.g., a lip of a patient 220. This radiant energy (hv₁ and hv₂) is shown traversing entirely through the adjacently placed tissue 200 and directly arriving at the detector 80. Accordingly the detector 80 detects a portion of this transmitted emitted energy (hv₁ and hv₂) and consequently produces a detector signal which can then be used to estimate the blood oxygen level in the tissue 200.

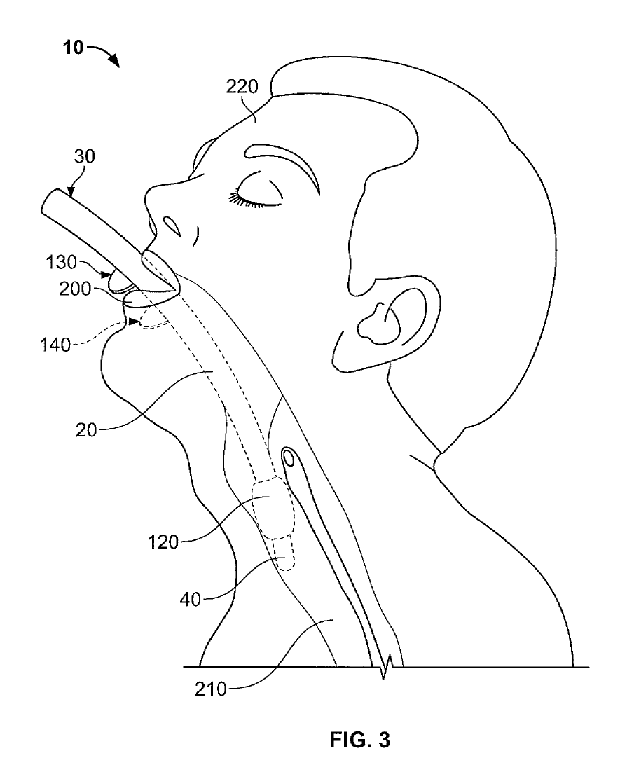

FIG. 3 depicts a perspective side view of an endotrachael-oximeter device 10 placed within a trachea of a patient 220 for measurement of blood oxygen levels in tissue 200. The flexible conduit 20 is shown having an upper end 30, a lower end 40, a ridge 130, a protrusion 140, an inflatable cuff 120.

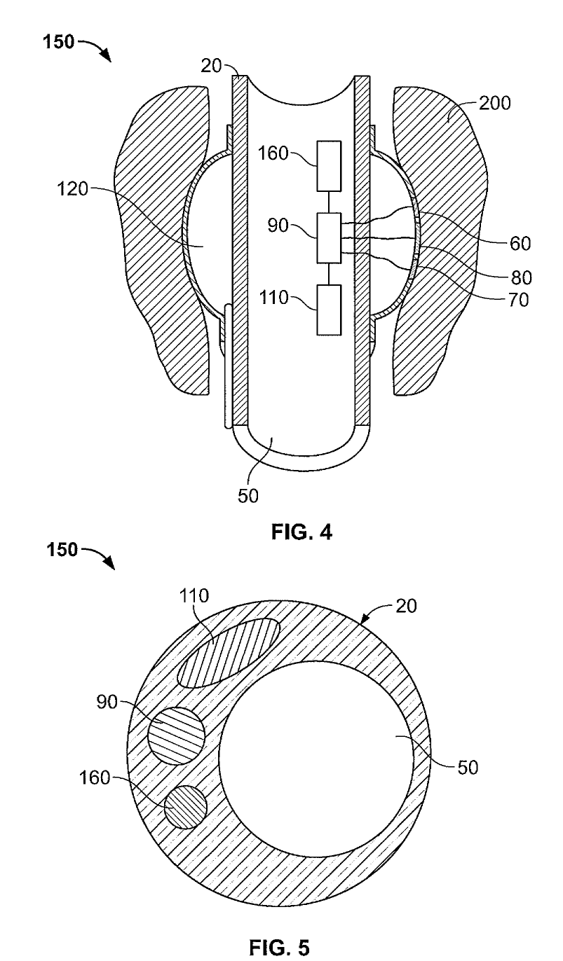

FIG. 4 depicts a partial cut away side view of an embodiment of the endotracheal-oximeter system 150 mounted within trachea. The flexible conduit 20 is shown having a lower end 40, an airway 50, an inflated cuff 120. Within the flexible conduit 20 is shown the control circuit 90, the power source 110, and the transmitter 160. The first and second EMF emitters (60 and 70) and the detector 80 are shown mounted to the inflated cuff 120 of the conduit 20. The first and second EMF emitters (60 and 70) and detector 80 are shown mounted onto the inflated cuff 120 with the conduit 20 secured in the tissue 200, e.g., trachea. In this manner the first and second EMF emitters (60 and 70) and detector 80 are intended to be in intimate contact with adjacent tissue 200. In this configuration the endotracheal-oximeter system 150 is intended to function in the reflectance oximeter operational mode. Accordingly an attending health care worker could conveniently obtain an estimate of the oxygen saturation levels in the tissue 200 of the patient 220 (not shown) by inspecting the display unit 100 at the monitor station 170 (not shown).

FIG. 5 depicts a partial cross-sectional view of the conduit 20 of another embodiment of the endotracheal-oximeter system 150. The control circuit 90, the power source 110 and the transmitter 160 are shown sequestering within the conduit 20.

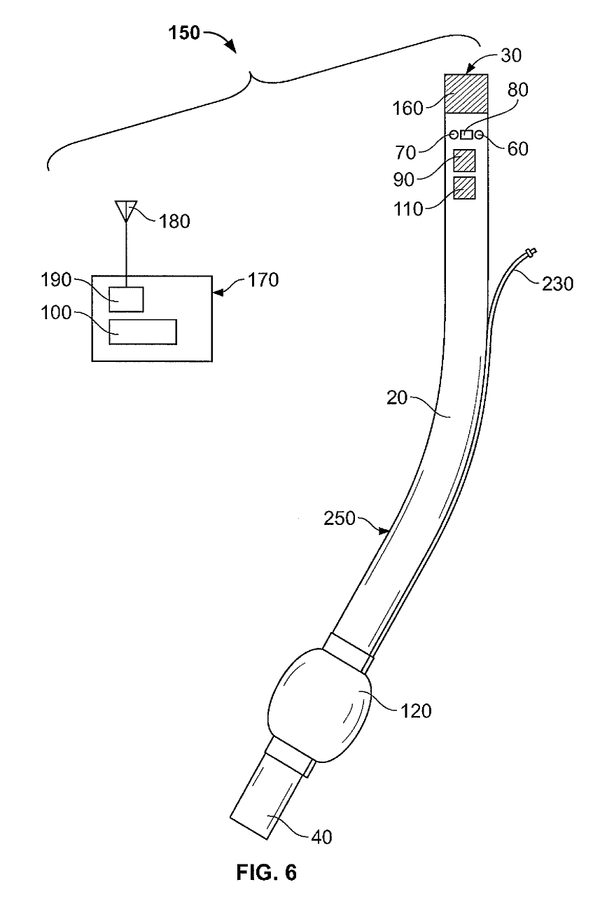

FIG. 6 depicts a side view of an embodiment of the endotracheal-oximeter system 150. The endotracheal-oximeter system 150 is shown having an endotracheal tube 240 and a monitor station 170. The endotracheal tube 240 is shown having a flexible conduit 20, a first EMF emitter 60, a second EMF emitter 70, a detector 80, a control circuit 90, a power source 110 and a transmitter 160. The monitor station 170 is shown having an antenna 180, a receiver circuit 190 and a display unit 100. The flexible conduit 20 is shown having an upper end 30, a lower end 40, an inflating tube 230, and an inflatable cuff 120. The first and second EMF emitters (60 and 70) and the detector 80 are shown attached to the conduit 20. The control circuit 90 attached to the conduit 20 in which the control circuit 90 is electrically coupled to the first EMF emitter 60, to the second EMF emitter 70, and to the detector 80. The power source 110 is shown attached to the conduit 20 in which the power source 110 is electrically coupled to the control circuit 90. The transmitter 160 is shown attached to the conduit 20 and is electrically coupled to the control circuit 90. The transmitter 160 is configured to transmit a broadcast signal proportionate to the detector signal from the control circuit 90 to the antenna 180 of the monitor station 170. The antenna 180 is configured to receive the broadcast signal transmitted from the transmitter 160. The receiver circuit 190 is electrically coupled to the antenna 180 in which the receiver circuit 190 is configured to process the received broadcast signal into a processed signal. The display unit 100 is operatively coupled to the receiver circuit 190 in which the display unit 100 is configured to display the processed signal which can be used as an estimate of oxygen saturation in tissue 200.

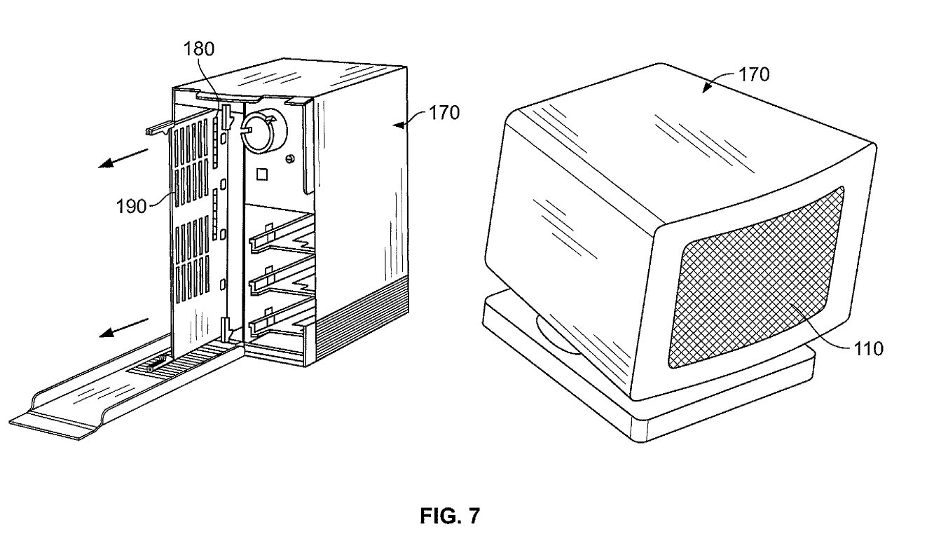

FIG. 7 depicts a perspective view of a monitoring station system 150 of an embodiment of the endotracheal-oximeter system 150. The monitoring station is shown having an antenna 180, a receiver circuit 190 and a display unit 100.

As to the manner of usage and operation of the present invention, the same should be apparent from the above description. Accordingly, no further discussion relating to the manner of usage and operation will be provided.

While preferred embodiments of the endotracheal-oximeter system device, system and method of using same has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Those skilled in the art will appreciate that the invention described herein is susceptible to variations and modifications other than those specifically described. It is to be understood that the invention includes all such variations and modifications that fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. An endotracheal-oximeter device comprising:
a flexible conduit comprising an upper end, a lower end, and an airway between the upper end and the lower end;
a first electromagnetic force (EMF) emitter attached to the conduit wherein the first EMF emitter is configured to emit a first EMF emission;
a second EMF emitter attached to the conduit wherein the second EMF emitter is configured to emit a second EMF emission;
a detector mounted directly onto the conduit so that the detector is optically aligned to receive the first and second EMF emissions, wherein the detector is configured to produce a detector signal in response to the received first and second EMF emissions;
a control circuit attached to the conduit, wherein the control circuit is electrically coupled to the first EMF emitter, to the second EMF emitter, and to the detector; and
a display unit attached to the conduit and electrically coupled to the control circuit, wherein the display unit is configured to provide an estimate of oxygen saturation in tissue in response to the detector signal; and
a power source attached to the conduit, wherein the power source is electrically coupled to the control circuit.

2. The device of claim 1 wherein the conduit comprises an inflatable cuff.

3. The device of claim 2 wherein the first EMF emitter, the second EMF emitter, and the detector are attached to the inflatable cuff of the conduit.

4. The device of claim 1 wherein the display unit comprises an audible display unit configured to produce an audible alarm when the estimate of oxygen saturation in blood is below a minimum threshold value.

5. The device of claim 1 wherein the display unit comprises a light emitting display unit.

6. The device of claim 1 wherein the conduit comprises a ridge having the first EMF emitter, the second EMF emitter, and the detector are attached to the ridge of the conduit.

7. The device of claim 1 wherein the conduit comprises a ridge having the first EMF emitter, the second EMF emitter are attached to the ridge and an opposing protrusion having the detector attached to the opposing protrusion.

8. The device of claim 1 wherein the control circuit and the power source are sequestered within the conduit.

9. The device of claim 1 wherein the first EMF emitter is configured to emit the first EMF emission at a red emission frequency between about 625 to about 740 nanometers.

10. The device of claim 9 wherein the first EMF emitter is configured to emit the first EMF emission at a mean red emission frequency of about 660 nanometers.

11. The device of claim 1 wherein the second EMF emitter is configured to emit the second EMF emission at an infrared emission frequency between about 800 to about 1100 nanometers.

12. The device of claim 11 wherein the second EMF emitter is configured to emit the second EMF emission at a mean infrared emission frequency of about 940 nanometers.

13. The device of claim 1 wherein the first and second EMF emitters are light emitting diodes (LEDs).

14. The device of claim 1 wherein the power source is selected from the group consisting of a battery power source and a high capacity capacitor power source.

15. The device of claim 1 wherein the detector is a photodetector.

16. An endotracheal-oximeter system comprising:
an endotracheal tube comprising:
a flexible conduit comprising an upper end, a lower end, and an airway between the upper end and the lower end;
a first electromagnetic force (EMF) emitter attached to the conduit wherein the first EMF emitter is configured to emit a first EMF emission;
a second EMF emitter attached to the conduit wherein the second EMF emitter is configured to emit a second EMF emission;
a detector mounted directly onto the conduit so that the detector is optically aligned to receive the first and second EMF emissions, wherein the detector is configured to produce a detector signal in response to the received first and second EMF emissions;
a control circuit attached to the conduit, wherein the control circuit is electrically coupled to the first EMF emitter, to the second EMF emitter, and to the detector; and
a power source attached to the conduit, wherein the power source is electrically coupled to the control circuit; and
a transmitter attached to the conduit and electrically coupled to the control circuit, wherein the transmitter is configured to transmit a broadcast signal proportionate to the detector signal from the control circuit; and
a monitor station comprising:
an antenna configured to be receive the broadcast signal transmitted from the transmitter;
a receiver circuit electrically coupled to the antenna, the receiver circuit is configured to process the received broadcast signal into a processed signal; and
a display unit operatively coupled to the receiver circuit, the display unit is configured to display the processed signal which can be used as an estimate of oxygen saturation in tissue.

17. The system of claim 16 wherein the first and second EMF emitters are light emitting diodes (LEDs).

18. The system of claim 16 wherein the power source is selected from the group consisting of a battery power source and a high capacity capacitor power source.

19. The system of claim 16 the detector is a photodetector.

20. A method of using an endotracheal-oximeter system comprising the steps of:
obtaining an endotracheal tube comprising:
a flexible conduit comprising an upper end, a lower end, and an airway between the upper end and the lower end;
a first electromagnetic force (EMF) emitter attached to the conduit wherein the first EMF emitter is configured to emit a first EMF emission;
a second EMF emitter attached to the conduit wherein the second EMF emitter is configured to emit a second EMF emission;
a detector mounted directly onto the conduit so that the detector is optically aligned to receive the first and second EMF emissions, wherein the detector is configured to produce a detector signal in response to the received first and second EMF emissions;
a control circuit attached to the conduit, wherein the control circuit is electrically coupled to the first EMF emitter, to the second EMF emitter, and to the detector; and
a power source attached to the conduit, wherein the power source is electrically coupled to the control circuit; and
a transmitter attached to the conduit and electrically coupled to the control circuit, wherein the transmitter is configured to transmit a broadcast signal proportionate to the detector signal from the control circuit; and
getting a monitor station comprising:
an antenna configured to be receive the broadcast signal transmitted from the transmitter;
a receiver circuit electrically coupled to the antenna, the receiver circuit is configured to process the received broadcast signal into a processed signal; and
a display unit operatively coupled to the receiver circuit, the display unit is configured to display the processed signal which can be used as an estimate of oxygen saturation in tissue;
activating the control circuit with the power source, wherein the activating step results in the first and second EMF emitters emitting their respective first and second EMF emissions so that the detector senses the emitted first and second EMF emissions and results in the transmitter transmitting the broadcast signal proportionate to the detector signal from the control circuit;
aligning a patient to receive the endotracheal tube;
sliding the lower end of the conduit of the endotracheal tube down into the trachae of the patient;
securing the endotrachael tube in the trachae of the patient;
forcing air through the airway of the conduit of the secured endotracheal tube to aerate the patient;
enabling the monitor station, wherein the enabling step enables the antenna to receive the broadcast signal transmitted from the transmitter so that the receiver circuit subsequently processes the received broadcast signal into a processed signal and display unit displays the processed signal; and
inspecting the displayed processed signal by using the displayed processed signal as an estimate of oxygen saturation in tissue of the patient.
